(19) United States
(12) Patent Application Publication
(10) Pub. No.: US 2025/0057702 A1
(43) Pub. Date: Feb. 20, 2025

(54) PRINTED NONWOVEN WEB WITH MICRODOT BOND PATTERN

(71) Applicant: FIBERTEX PERSONAL CARE A/S, Aalborg Ost (DK)

(72) Inventors: Florian HAHNE, Luttgenrode (DE); Jesper JACOBSEN, Klarup (DK); Brian UDENGAARD, Lystrup (DK)

(73) Assignee: FIBERTEX PERSONAL CARE A/S, Aalborg Ost (DK)

(21) Appl. No.: 18/723,479

(22) PCT Filed: Mar. 2, 2023
(86) PCT No.: PCT/EP2023/055260

§ 371 Date: Jun. 24, 2024

(30) Foreign Application Priority Data
Mar. 2, 2022 (EP) 22159685.1

(51) Int. Cl. A61F 13/15; B41M 3/00; D04H 3/14; D04H 3/16

(52) U.S. Cl. CPC A61F 13/15731; A61F 13/15203; B41M 3/00; D04H 3/14; D04H 3/16; A61F 2013/15243

(57) ABSTRACT

The invention relates to printed nonwoven webs comprising a pattern of fusion-embossed bonding points and being printed. The invention further relates to a method for making such nonwoven webs, and to uses for such nonwoven webs.

The invention relates to nonwoven webs comprising a pattern of fusion-embossed bonding points on at least one of its surfaces and being printed on the same surface. The invention further relates to a method for making such nonwoven webs, and to uses for such nonwoven webs.

Hygiene industry uses nonwoven webs as materials for making products such as baby diapers or adult incontinence products on a large scale. Webs comprising or consisting of spunbonded nonwoven material and bonded by calender embossing have become industry standard for many applications. These webs not only have desirable product characteristics, they can also be economically produced at high line speeds. Depending on the type of application, the materials must be printed with texts, graphical patterns or images. Most typically, printing these materials is facilitated by flexographic ink printing, where the nonwoven web is fed to a flexographic printing unit at high translation speed and where ink is transferred from a print roll directly to the surface of the web.

The image quality when ink-printing a calender-bonded nonwoven web comprising a pattern of fusion-embossed bonding points on its printed surface is limited, however. The reason is that the bonding points take on a higher color intensity and contrast than the remaining parts of the fabric, which makes the print appear pixelated and inhomogeneous.

The present invention aims to improve image quality for prints on calender-bonded nonwoven webs without detriment to production efficiency and economy.

Against this background the invention proposes a printed nonwoven web comprising a pattern of fusion-embossed bonding points, wherein the nonwoven web is ink-printed on the surface showing the patter of bonding points, wherein the pattern of bonding points fulfils the following conditions: (a) the number of bonding points on the surface is greater than 70/cm², preferably greater than 75/cm²; (b) the average area size of the individual bonding points is smaller 1.5 mm², preferably smaller than 1.35 mm²; and (c) the average distance between bonding points, from centre to centre, is smaller than 1.5 mm, preferably smaller than 1.3 mm. The bonding pattern is preferably homogenous over the entire surface of the fabric.

The conditions are representative of using more and smaller bonding points than previously used in the art. The smaller bonding points lead to a surface with a more uniform appearance. This is especially advantageous for subsequent printing as it makes the print appear more uniform. The reason for this is that wherever a part of the printed design includes a bonding point, that area will appear to be slightly differently coloured than the unbonded printed surface due to the differences in smoothness and opacity. With the smaller bonding points and a higher density bonding pattern this becomes less pronounced, so that the printed design looks better.

In one embodiment, the bond area, defined as fraction of the total area of the surface that is occupied by the bonding points, is smaller than 18%, preferably smaller than 15%, more preferably than 12%, and yet more preferably smaller than 11%. The smaller the overall bond area, the smaller the overall area with slightly difference colouring. Hence, and overall smaller bond area can also contribute to better print quality.

The respective printed nonwoven webs can be produced by a method, wherein a nonwoven web is bonded by calender embossing to form a nonwoven web comprising a pattern of fusion-embossed bonding points fulfilling the above conditions on at least one of its surfaces, and wherein the nonwoven web is ink-printed on the surface showing the patter of bonding points.

Preferably, the ink printing is flexographic ink printing. In a flexographic ink printing unit, the nonwoven web is typically passed run across the surface of a print roll. The print roll comprises an exchangeable cylindrical sleeve folded around a core cylinder. The sleeve comprises a mirrored master of the required image as a positive 3D relief. Ink is transferred from an ink reservoir to the print roll from an anilox roll, which has surface cells for dosing the ink. Doctor blades scrape off excessive ink from outside the cells. The print roll comprises a mirrored master of the required image as a positive 3D relief.

Alternatively, the ink printing can also be ink jet printing. This printing method may not allow the same line speeds as flexographic printing, but is more flexible in terms of individualization or changes. Digital prints can be realized with ink jet printing.

The ink can be a water-or solvent-based ink and the surface can be dried after ink application. Alternatively, the ink can be a UV-curable ink that contains reactive monomers and/or oligomers, pigments and photoinitiators, in which the surface can be irradiated with UV light after the ink application. UV-curable inks can be advantageous from a standpoint of increased adhesion and reduced VOC emission.

The inventively configured bonding point pattern can be operated in a large range of print resolutions. Specifically, the contact areas on the print roller are typically dotted and the dots are arranged in lines. The line screen, as expressed in lines per cm, is representative for print resolution. In one embodiment, the line screen can be between 15 L/cm and 50 L/cm. Print resolutions of greater than about 30 L/cm could be used to obtain superior print quality.

In one embodiment, the nonwoven web is a spunbonded or spunmelt nonwoven material. Preferably, the material is formed from polyolefin fibers. Suitable polyolefins comprise, for example, polypropylene (PP), polyethylene (PP), or copolymers comprising propylene-or ethylene-based copolymers (co-PP; co-PE). A spunbonded nonwoven material may be combined with additional same or different spunbonded(S) layers and/or meltblown (M) layers to obtain multiple layer webs of SS, SMS or similar types.

During bonding the material is run across the surface of a calender roll having embossing projections on the surface that fulfil the conditions laid out above. In a preferred embodiment, bonding comprises ultrasonic bonding, where ultrasonic vibrations are introduced into the embossing projections and the fibers are fused together by ultrasonic fusion. Other embodiments use thermal bonding where the embossing projections are heated and the fibers are fused together by thermal fusion.

For optimum production efficiency, the method steps of nonwoven material formation by fiber laydown and bonding on the one hand and by printing on the nonwoven on the other hand are preferably carried out inline. Alternatively, for greater flexibility, an offline setting where a prefabricated nonwoven material is printed separately can be used.

In general, the line speed for nonwoven material formation and printing, be it together in an inline setting or separately in an offline setting, is preferably at least 100 meters per minute, more preferably greater than 150 meters per minute, and even more preferably greater than 200 meters per minute.

The printed nonwoven webs of the invention can be used in hygiene articles such as baby diapers, sanitary napkins, or adult incontinence products. Alternatively, the nonwoven webs can also be used in medical applications such as gowns, face masks, nursing pads, or bed sheets. Further, the nonwoven webs can be used in household products like wipes and cleaning towels.

By reducing the distance between the bonding points, the quality of the print gets very close to what can be achieved for other nonwoven types bonded without creating bonding points, like on air through carded, since the bond pattern becomes less pronounced. The bonding dots are so small that they do not interfere with the quality of the print. At the same time, production efficiency and product quality remains high. Specifically, the calander bonded nonwovens have better physical properties than nonwovens bonded by other methods like air through bonding.

Further details and advantages of the invention will become apparent from the figures and examples described in the following. The figures show:

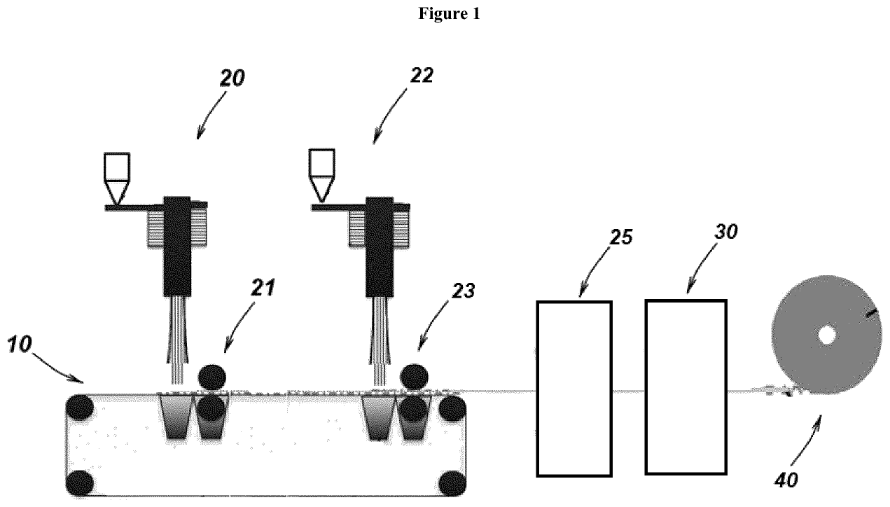

FIG. 1: a schematic setup of a production line for making printed nonwoven webs according to the invention;

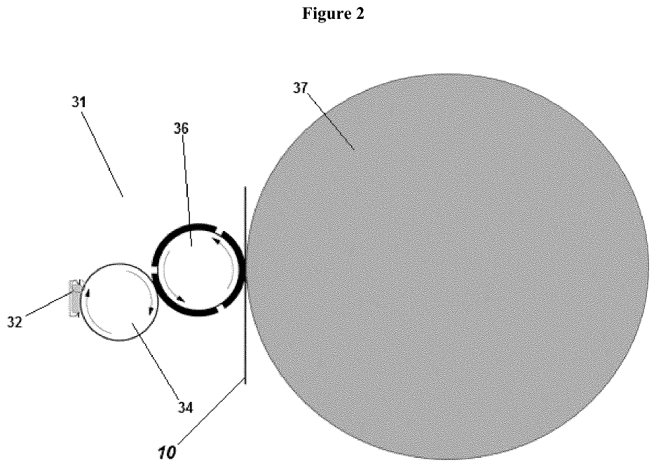

FIG. 2: an illustration showing the principle of flexographic ink printing;

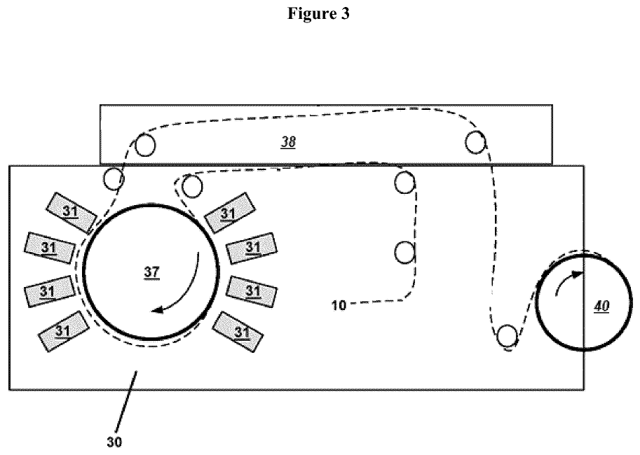

FIG. 3: a typical flexographic ink printing layout used in nonwoven printing;

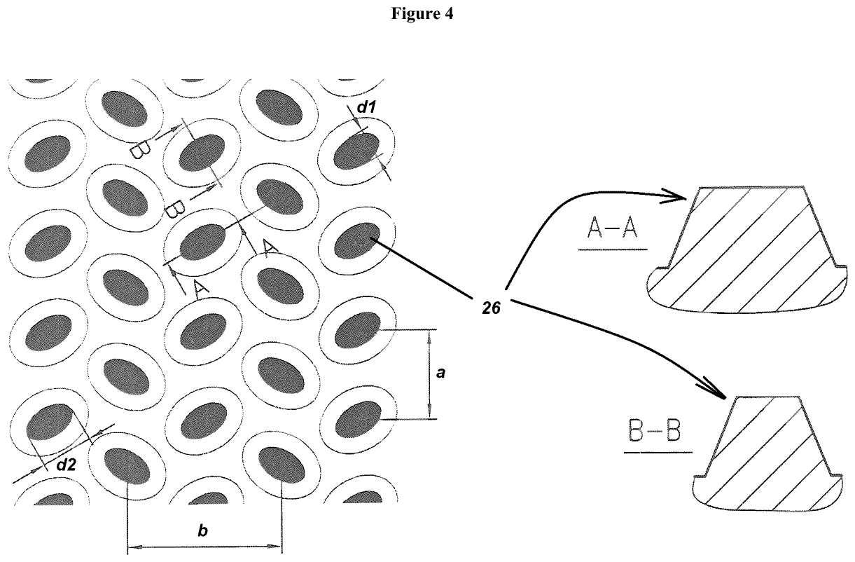

FIG. 4: a bonding pattern as used in the prior art; and

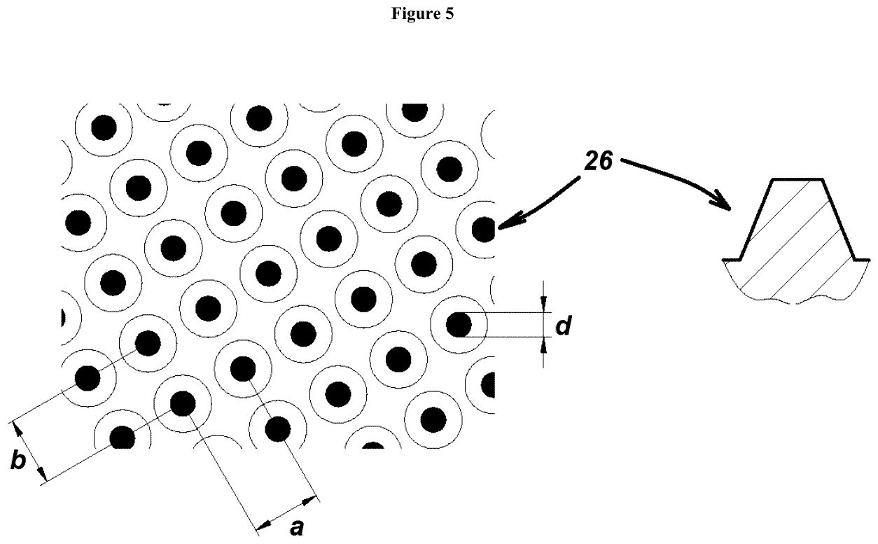

FIG. 5: a bonding pattern according to the invention.

An exemplary machine setup for making printed nonwoven web 10 according to the invention is shown in FIG. 1.

The setup comprises a conveyor belt and two spunbonding machines 20, 22 arranged in line on the conveyor belt. In each of the spunbonding machines, a molten thermoplastic polymer is extruded through the holes of a die. The extruded fiber strands are then quenched and drawn/stretched to form endless fibers, which are then laid onto the conveyor belt or, in case of the second laydown, on the first web that has been previously deposited thereon. Each of the spunbonding machines 20, 22 is followed by a pair of pre-compaction rollers 21, 23 for pre-compacting the respective webs.

The pre-compacted web is then bonded by calander-embossing in a bonding unit 25, where a fabric is run over the surface of a calander roll having a pattern of embossing projections on its surface. Thermal heat or ultrasonic energy is introduced into the projections by a source of thermal or ultrasonic energy and the web is fusion bonded at the respective locations to obtain a pattern of bonding points that corresponds to the pattern of projections on the calander roll surface.

The bonding is followed by a printing step in printing unit 30, which will be described in more detail in the following. The product web is finally collected on the product roll 40.

FIG. 2 shows a flexographic printing unit 31 comprising a manifold for transferring ink onto the surface of an anilox roll 34. The manifold comprises an ink reservoir 32 and doctor blades acting as retaining and scrap blades. The surface of the anilox roll 34 comprises cells that enable dosing the ink to be transferred to the print roll 36. The surface of the print roll 36 comprises a positive mirrored master of the required image as a 3D relief. The print roll 36 transfers ink to the nonwoven web 10 while it is pressed against the print roll 36 by a counter-rotating impression drum 37.

As shown in FIG. 3, multiple units 31 comprising manifolds 33, anvil rolls 34 and print rolls 36 are arranged around a common impression drum 37 for multi-color printing in a printing unit 30. A drying or curing unit 38 follows inline.

FIG. 4 shows a pattern of embossing projections on the surface of a calander roll, that has been used for producing webs of the generic kind in the past. It comprises elliptically shaped embossing projections 26 that have different lengths d1 and widths d2. The bond area is approx. 0.25 mm². The distances a and b between the projections 26 are greater 1.5 and greater 2.5, respectively, and are such that the number of projections per area is approx. 50 and the bonding area, meaning the fraction of the total surface area occupied by the flat projection surfaces, is approx. 18%.

FIG. 5 shows a pattern that is according to the invention. It is symmetrical and comprises circular embossing projections of a diameter d that is 0.4 mm. The bond area is hence approx. 0.125 mm². The pitch a (=b) between the bonding points is 1.12 mm. This results in a number of bonding points per area of 80/cm² and a total bonding area of 10%.

### EXAMPLES

The examples compare the print quality on inventive microdot material and standard bonded nonwoven materials. All tests were carried out on the same 17 g/m² polypropylene three-layer spundbond (SSS) standard nonwoven material. The samples in the comparative tests were bonded using a bond pattern as in FIG. 4. The samples in the inventive tests were bonded using a bond pattern as in FIG. 5.

The samples all contained exactly the same print image and were printed, by flexographic ink printing, under exactly the same conditions (screen, sleeves, inks used, press, etc.).

The measurement procedure was as follows: In the first step, the exactly identical areas of the print samples to be compared are glued onto a white sheet of paper and scanned with a scanner (min. 300dpi resolution) as a PDF. The PDFs are then each opened in the software Adobe Photoshop 2023 and adjusted exactly to the area to be measured, before being converted to grayscale. Then, a gradation step is applied to separate the darker uneven areas from the even ones. The pixels are displayed in black after the gradation curve has been applied. For this purpose, a fixed sensitivity is set so that color tones with different brightness values can be measured. For the sensitivity setting, the L-value of the darkest shade of the area to be measured is determined using a measuring device (X-Rite Exact). The default sensitivity value (input value) with an L value of 75 is 48. If the L value deviates from +1, the sensitivity value decreases by 2. If the L value deviates from −1, the sensitivity value increases by 2.

Finally, with the help of the histogram, it can be read off how many deviating black pixels are present in the print area, and the values can be compared between the two types of materials.

In all pressure ranges with an L value below 85, the inventive materials have less deviation than the comparative materials. This has been found for four different pictures P1-P4 that are print designs used in the absorbent hygiene industry. The results are shown in Table 1 below.

TABLE 1

| Picture | P1 | P2 | P3 | P4 |
|---|---|---|---|---|
| Total printed pixels | 7.777.244 | 11.331.414 | 1.471.901 | 6.436.458 |
| Black pixels comparative | 89.520 | 290.296 | 18.090 | 124.389 |
| sample | | | | |
| Ratio of black pixels in | 1.2% | 2.6% | 1.2% | 1.9% |
| comparative sample | | | | |
| Black pixels inventive sample | 40.370 | 13.825 | 11.800 | 62.425 |
| Ratio of black pixels in | 0.5% | 0.1% | 0.8% | 1.0% |
| inventive sample | | | | |
| Sensitivity | 48 | 72 | 58 | 48 |
| Total pixels | 10.164.610 | 11.331.414 | 8.076.819 | 8.958.048 |
| Darkest L Value | 75 | 63 | 70 | 75 |

It has been found for a number of different solid single color prints (rectangular). Corresponding results are given in Table 2 below.

TABLE 2

| Color | M100 | C100 | K100 | MY100 |
|---|---|---|---|---|
| Total printed pixels | 254.000 | 254.000 | 254.000 | 254.000 |
| Black pixels comparative | 9.390 | 14.821 | 6.327 | 5.880 |
| sample | | | | |
| Ratio of black pixels in | 3.7% | 5.8% | 2.6% | 2.3% |
| comparative sample | | | | |
| Black pixels inventive sample | 3.771 | 7.088 | 3.760 | 2.439 |
| Ratio of black pixels in | 1.5% | 2.8% | 1.5% | 1.0% |
| inventive sample | | | | |
| Sensitivity | 44 | 46 | 48 | 48 |
| L Value | 77 | 76 | 75 | 75 |

| Color | YC100 | CM100 | P2593 100 |
|---|---|---|---|
| Total printed pixels | 254.000 | 254.000 | 254.000 |
| Black pixels comparative | 7.660 | 10.412 | 10.612 |
| sample | | | |
| Ratio of black pixels in | 3.1% | 4.1% | 4.2% |
| comparative sample | | | |
| Black pixels inventive sample | 4.670 | 4.579 | 3.020 |
| Ratio of black pixels in | 1.8% | 1.8% | 1.2% |
| inventive sample | | | |
| Sensitivity | 50 | 66 | 54 |
| L Value | 74 | 66 | 72 |

1-11. (canceled)

12. A printed nonwoven web comprising a pattern of fusion-embossed bonding points, wherein the nonwoven web is ink-printed on the surface showing the pattern of bonding points, wherein
the pattern of bonding points is homogenous over the entire surface of the fabric and fulfills the following conditions:
(a) the number of bonding points on the surface is greater than 70/cm²;
(b) the average area size of the individual bonding points is smaller than 1.5 mm²; and
(c) the average distance between bonding points, from centre to centre, is smaller than 1.3 mm.

13. The printed nonwoven web according to claim 12, wherein a bond area, defined as fraction of the total area of the surface that is occupied by the bonding points, is smaller than 18%.

14. The printed nonwoven web according to claim 12, wherein the web is a spunbonded or spunmelt nonwoven web.

15. A method for making a printed nonwoven web, said method comprising a nonwoven web that is bonded by calender embossing to form a nonwoven web comprising a pattern of fusion-embossed bonding points, and wherein the nonwoven web is ink-printed on the surface showing the pattern of bonding points, wherein the pattern of bonding points is homogenous over the entire surface of the fabric and fulfills the following conditions:
(a) the number of bonding points on the surface is greater than 70/cm²;
(b) the average area size of the individual bonding points is smaller than 1.5 mm²; and
(c) the average distance between bonding points, from centre to centre, is smaller than 1.5 mm.

16. The method according to claim 15, wherein a bond area, defined as fraction of the total area of the surface that is occupied by the bonding points, is smaller than 18%.

17. The method according to claim 15, wherein the ink printing is flexographic ink printing.

18. The method according to claim 17, wherein a line screen is between 15 L/cm and 50 L/cm.

19. The method according to claim 15, wherein the ink printing is ink jet printing.

20. The method according to claim 15, wherein the web is a spunbonded nonwoven web.

21. The method according to claim 15, wherein the calender embossing comprises ultrasonic embossing.

22. A method to manufacture hygiene articles, said method comprising utilizing the printed nonwoven web of claim 12 in said hygiene articles.

23. The printed nonwoven web according to claim 12, wherein (a) the number of bonding points on the surface is greater than 75/cm²;
(b) the average area size of the individual bonding points is smaller than 1.35 mm²; and
(c) the average distance between bonding points, from centre to centre, is smaller 1.3 mm.

24. The printed nonwoven web according to claim 12, wherein a bond area, defined as fraction of the total area of the surface that is occupied by the bonding points, is smaller than 15%.

25. The printed nonwoven web according to claim 12, wherein a bond area, defined as fraction of the total area of the surface that is occupied by the bonding points, is smaller than 12%.

26. The printed nonwoven web according to claim 12, wherein a bond area, defined as fraction of the total area of the surface that is occupied by the bonding points, is smaller than 11%.

27. The method of claim 15, wherein (a) the number of bonding points on the surface is greater than 75/cm²;
(b) the average area size of the individual bonding points is smaller than 1.35 mm²; and
(c) the average distance between bonding points, from centre to centre, is smaller than 1.3 mm

28. The method according to claim 15, wherein a bond area, defined as fraction of the total area of the surface that is occupied by the bonding points, is smaller than 15%.

29. The method according to claim 15, wherein a bond area, defined as fraction of the total area of the surface that is occupied by the bonding points, is smaller than 12%.

30. The method according to claim 15, wherein a bond area, defined as fraction of the total area of the surface that is occupied by the bonding points, is smaller than 11%.

31. The method to manufacture hygiene articles according to claim 22, wherein said hygiene articles are diapers.
